(19) United States
(12) Patent Application Publication
(10) Pub. No.: US 2002/0102596 A1
(43) Pub. Date: Aug. 1, 2002

(54) Methods for detecting interaction of molecules with surface-bound reagents

(76) Inventor: Lloyd Mervyn Davis, Tullahoma, TN (US)

Correspondence Address: Richard T. Redano, Duane Morris LLP, Suite 500, One Greenway Plaza, Houston, TX, 77046, US

(21) Appl. No.: 10/066,074

(22) Filed: Jan. 31, 2002

Related U.S. Application Data
(60) Provisional application No. 60/265,257, filed on Jan. 31, 2001.

(51) Int. Cl. C12Q 1/68

(52) U.S. Cl. 435/6; 435/287.2; 422/82.08; 436/56; 436/172

(57) ABSTRACT

Methods and devices for detecting the interaction of single molecules with surface-bound reagents are disclosed. The methods utilize synchronized techniques for controlling the interaction between labeled molecules in a flowing solution with reagents supported on a surface, and incorporate optical detection techniques such as fluorescence measurements. One application of the disclosed methods and devices is the detection of chemical reactions in which the nucleobases of labeled NTP molecules become incorporated into the DNA of a surface-bound enzyme-DNA complex with release of the labels.

### BACKGROUND OF THE INVENTION

The ability to optically detect or image individual labeled molecules in solution generally depends upon limiting the concentration of labeled molecules to a concentration at which the statistical probability of having more than one labeled molecule within a volume that is optically-resolvable (“detection volume”) is much less than the statistical probability of having just one molecule within the detection volume. Such ability to optically detect individual labeled molecules in solution is particularly applicable to genotyping and/or sequencing nucleic acids.

U.S. Pat. Nos. 4,793,705 and 4,979,824 disclose methods for single-molecule imaging and detection in solution. In general, the ability to optically detect or image individual labeled molecules in solution requires that the concentration of labeled molecules be limited to a concentration at which the statistical probability of having more than one labeled molecule within a volume that is optically resolvable (“detection volume”) is much less than the statistical probability of having just one molecule within the detection volume. Such ability to optically detect individual labeled molecules in solution is applicable to methods that require detecting individual chemical reactions or interactions, and is particularly applicable to methods for genotyping and/or sequencing nucleic acids, such as that disclosed in U.S. Pat. No. 6,255,083.

In bulk solution, the smallest detection volume achievable with a far-field optical system is determined by the numerical apertures for excitation and collection of light. For example, volumes of about 3×10⁻¹⁹ m³ (about 0.5 microns in diameter and about 1.5 microns in length) can be achieved in confocal epi-illumination set-ups for single molecule detection. A volume of about 4×10⁻²⁰ m³ (0.5 microns in diameter, 0.2 microns in length) can be obtained in a microfluidic flow cell of thickness 0.2 microns. Smaller volumes can be achieved by use of near-field optical systems for excitation and/or emission, but such systems have other disadvantages. If the statistical probability for having two or more molecules in the detection volume is 5% of the statistical probability for having just one molecule, and if molecules are randomly distributed, then the Poisson distribution can be used to show that the probability for having just one molecule must be 0.1. For a volume of 4×10⁻²⁰ m³, this probability corresponds to a concentration of 2.5×10¹⁸ molecules m⁻³, or 4×10⁻⁹ moles per liter (“M”). At such low concentrations, it is believed that most chemical reactions proceed at a slow or sub-optimal rate limited by diffusion. Further, the individual reactions then occur at random times, determined mostly by the statistical times at which individual reactants happen to diffuse together.

For the sequencing of nucleic acids, such as DNA and RNA, by synthesis, according to Michelis-Mentin enzyme incorporation kinetics, the molar concentration at which the nucleobase incorporation rate is about half of its maximum rate is typically 10⁻⁵M, so that the reaction rate for a concentration of 4×10⁻⁹ M is less than 0.04% of the maximum rate. Assuming a maximum reaction rate of 300 s⁻¹, at maximum, one reaction would occur each 8.3 seconds. A large increase in the reaction rate above this value is necessary if DNA sequencing by synthesis is to be competitive with conventional sequencing approaches. For example, Perkin Elmer's current ABI Prism 3700 DNA sequencer can provide sequencing information at a rate of 5 bases per second, and a further order of magnitude increase in speed can be anticipated in future machines. For one reaction each 8.3 seconds, hundreds of parallel channels on a sequencing-by-synthesis machine would be required just to equal the sequencing rate of the conventional approach.

Sequencing or genotyping of nucleic acids, such as DNA and RNA, by single nucleic acid synthesis, is disclosed in U.S. Pat. No. 6,255,083. Incorporation reactions of nucleobases into a target nucleic acid by a polymerase are detected by using labeled nucleobases and by detecting the individual labels that are released from the nucleobases upon incorporation. However, in order to detect or image individual labeled nucleobases, either the concentration of labeled nucleobases must be sufficiently dilute that the labels can be individually optically resolved, or otherwise the label must give a distinguishable signal after it is released from the nucleobase, so that only those labels released following incorporation events are detected. U.S. Pat. Nos 6,232,075 and 6,306,607 disclose methods and kits for covalent attachment of fluorescence quenching molecules to the fluorescently labeled nucleobase molecules, to enable labels to differentially fluoresce when they are released following an incorporation event. However, this approach complicates the chemistry, and furthermore, the quenching molecule may interfere with the mechanics of the polymerase molecule as it attempts to incorporate a nucleobase that has both a label and a quenching molecule attached. Also, incomplete quenching, or incomplete differentiation of the fluorescence from labels of labeled nucleobases prior to incorporation, results in a background that dictates the use of a low concentration of labeled nucleobases, and hence a low rate for incorporation. If quenching molecules were not used, then all labeled nucleobases would present visible signals and in order to individually detect or image the labeled nucleobases, the concentration of labeled nucleobases would need to be at a level of about 4′10-9 M or lower. The incorporation reaction would then proceed at a slow or sub-optimal rate limited by diffusion.

Patent publication PCT WO 01/94609 A1 discloses an alternative approach to cause the label to give a distinguishable signal after it is released following incorporation. In this case the released label has a different electrical charge and may be electrophoretically separated from other labeled nucleobases. However, this approach also complicates the chemistry, and the electrical charges may similarly interfere with the mechanics of the polymerase molecule.

A need remains for methods for detecting individual reactions or interactions involving single molecules, particularly for such applications as nucleic acid sequencing and genotyping. Methods that enable detecting individual reactions or interactions of single molecules with other agents, molecules, or complexes at higher reaction rates and/or with improved sensitivity are particularly desirable. Methods that enable the times of occurrence of individual reactions or interactions to be controlled are also desirable. The present invention is directed to these, and other, important ends.

### SUMMARY OF THE INVENTION

The present invention provides methods and devices for optically detecting single labeled molecules that have participated in certain types of chemical reactions and interactions, with surface bound reagent molecules. It also provides methods for individually detecting certain types of chemical reactions and interactions. Chemical reactions and interactions in which such single labeled molecules are involved that can be detected using the methods of the invention include reactions having at least one intermediate or final step in which the label of the labeled molecule is bound or entrapped by the surface-bound reagent.

The invention provides methods and devices for detecting individual reactions or interactions involving labeled molecules in which the concentration of labeled molecules can be about 10⁻⁵ M, or higher. Conventional single molecule detection methods will not work at such high concentrations, because individual labeled molecules can no longer be spatially resolved. The ability to detect individual interactions when using labeled molecules at concentrations of about 10⁻⁵ M or higher can allow the detection of reactions that occur at higher reaction rates to the point at which the rate is no longer limited by diffusion.

Furthermore, the methods and devices of the invention enable one skilled in the art to choose operating parameters for controlling the time between individual chemical reactions when individual chemical reactions occur repeatedly. The possibility of missing the detection of a reaction due to two or more reactions occurring in quick succession is thereby minimized by the methods disclosed herein.

One aspect of the present invention is a method for optically detecting the chemical reaction of a single labeled molecule with a reagent supported on a surface, wherein the chemical reaction involves at least one step in which the label of the labeled molecule is bound or entrapped by the surface-bound reagent. The method includes:

a) providing a flow cell:

b) providing within said flow cell a solid support having a surface;

c) supporting at least one reagent molecule to said surface;

d) introducing at least two flowing solutions into said flow cell, wherein at least one solution comprises a labeled molecule that can contact said reagent molecule, and at least one solution comprises buffer with no detectable labels, and wherein the at least two solutions are at different locations within the flow cell at any time;

e) immersing the supported reagent in a solution comprising labeled molecules;

f) switching or directing the flowing solutions with respect to the supported reagent, or switching the location of the supported reagent with respect to the flowing solutions, to immerse the supported reagent in the solution comprising buffer with no labeled molecules;

g) providing a light source for illuminating an illumination zone within the buffer solution;

h) providing a detector for detecting light emitted from the illumination zone;

i) substantially simultaneously to said switching or directing the flowing solutions with respect to the supported reagent, or switching the location of the supported reagent with respect to the flowing solutions, switching at least one of said light source, detector, or location of said supported reagent to cause the label of a labeled molecule which has reacted with said supported reagent to pass through said illumination zone; and

j) detecting light emitted at said illumination zone to detect the presence of a labeled molecule.

In preferred embodiments, after the supported reagent is immersed in a solution comprising labeled molecules, the label of a labeled molecule is bound or entrapped nearby the supported reagent for a time interval of at least about 100 microseconds. The time interval is referred to herein as Δt. Also preferably, the switching of the flow in step (f) and the substantially simultaneous switching in step (i) are executed after an interval of time that is less than Δt.

In preferred embodiments, the label is a fluorescent moiety or a fluorescent molecule. In particularly preferred embodiments, the reagent molecule is a target nucleic acid and a primer nucleic acid which complements a region of the nucleic acid and at least one nucleic acid polymerase, and the labeled molecule is a labeled nucleotide triphosphate (NTP). In other particularly preferred embodiments the label is located on the gamma or beta phosphate of a NTP.

In some preferred embodiments, the reagent molecule is a single-stranded target nucleic acid and the reagent molecule is a fluorescently-labeled complementary nucleic acid strand.

In some preferred embodiments, the reagent molecule is a cell-signaling receptor on the surface of a cell, the solid support is the cell itself, and the reagent molecule is a fluorescently-labeled ligand.

In preferred embodiments, the solution comprising labeled molecules is at a concentration of about 10⁻⁷M or more. More preferably, the solution concentration is about 10⁻⁶M or more.

In some preferred embodiments of the invention, two or more distinguishable types of labels are used to label two or more different types of reactive molecules. In preferred embodiments, the labels are optically detected and the optical detection of the labels includes determining which type of label is detected. Labels can be distinguished by, for example, color of excitation light or emission light, fluorescence brightness, fluorescence lifetime, electrophoretic mobility, or other properties of the labels, or the location or time of detection. In some embodiments, an array of supported reagents is used and optical detection of the reaction is separately accomplished for each reagent of the array.

Another aspect of the present invention is a system for optically detecting the chemical reaction of a labeled molecule with a surface-bound reagent, wherein the chemical reaction involves at least one step in which the label of the labeled molecule is bound or stereo-chemically entrapped by the reagent, at least for a short interval of time Δt exceeding about 100 microseconds, comprising

a. a surface to which is bound a single reagent molecule, or an array of single reagent molecules at resolvable locations, said surface being contained within a flow cell;

b. an illumination zone;

c. a light source for illuminating said illumination zone;

d. an optical detector for detecting light emitted by a label on said labeled molecule.;

e. at least two flowing solutions, wherein at least one solution comprises labeled molecules that can react with said surface-bound reagent, and at least one solution comprises buffer with no detectable label, and wherein the two solutions exist at distinct locations within said flow cell;

f. a means for switching at least one of the flow of said solutions, the physical location of the surface of said surface-bound reagent, to immerse the surface-bound reagent molecule in only one solution at a time;

g. a means for switching on and off at least one of the light source, the detection of light, or the relative location of the zone of illumination-and-detection and the surface of the surface-bound reagent, so that labels that pass nearby to the surface either pass through said zone or do not pass through said zone; and

h. a circuit for effectively synchronizing said illuminating said illumination zone with said switching at least one of the flow of said solutions or the physical location of the surface of said surface-bound reagent, to immerse the surface-bound reagent molecule in only one solution at a time.

### BRIEF DESCRIPTION OF THE DRAWINGS

FIG. 1 is a schematic representation of a system according to a preferred embodiment for detecting the reaction of a NTP in solution with an affixed enzyme-containing reagent.

FIG. 2 is a schematic representation of a preferred embodiment utilizing automatic synchronization of the switching of the concentration of a flowing solution in which a reagent is immersed, with the switching of detection location.

### DETAILED DESCRIPTION

In many reactions and interactions between labeled molecules and reactant molecules, the label becomes temporarily supported at the site of the reactant molecule, and during this time it is not free to be carried away by diffusion or flow. For example, a large class of chemical interactions, particularly those involving complex biological molecules, involve a series of intermediate steps, some of which require motion of parts of the molecules or other events that require a minimum time to be executed, and during this time, the labeled reactant molecule is temporarily bound to the reagent by temporary covalent bonds, hydrogen bonds, or entrapment of a part of the labeled molecule. In many other chemical reactions, the label of the labeled reactant molecule becomes affixed to the reagent molecule permanently during the course of the reaction.

The present disclosure provides methods and systems for optically detecting or imaging a label of a single labeled molecule that has participated in a chemical reaction with a surface-bound reagent molecule. The major advantage of the disclosed method is that the single labeled molecule is provided from a solution that is of a sufficiently high concentration that the reaction between labeled molecules and the immobilized reagent occurs very quickly, i.e., the rate at which the reaction occurs is faster than that obtained with label concentrations that would allow single labeled molecules to be optically resolved, and it can be so fast that the reaction rate is no longer dominated by the diffusional rate at which labeled molecules encounter the immobilized reagent. The disclosed method is preferably utilized for detecting a chemical reaction or interaction that involves a step in which the label of a labeled molecule is bound or entrapped by the immobilized reagent for a duration Δt in excess of about 100 microseconds.

The methods disclosed herein are useful for genotyping and/or sequencing nucleic acids, including DNA and RNA. While “genotyping” is the determination of the identification of the nucleobases in a nucleic acid, “sequencing” also includes the determination of the order and position of the nucleobases. Detection of a chemical reaction of incorporation of a nucleobase into an immobilized complex by use of a flow cell with two illumination zones, one upstream and one downstream from the immobilized complex is disclosed in copending patent application serial no. ______ , filed on Jan. 29, 2002, and incorporated herein by reference in its entirety, which claims priority to Provisional Patent Application serial no. 60/264,790, entitled “Method for detection of incorporation of a single nucleotide onto a nucleic acid primer by a polymerase enzyme,” filed Jan. 29, 2001, also incorporated herein by reference.

Unless otherwise stated, the terms below defined, when used herein, have the definitions set forth herein.

“Nucleobase” means one of the purine or pyrimidine derivatives that are components of nucleotides of nucleic acids, i.e. adenine, thymine, guanine, cystein, uracil.

“Nucleotide” means a structural unit of a nucleic acid, which is an ester of a nucleoside and phosphoric acid.

“Nucleoside” means a glycoside, comprising a pentose sugar linked to a purine or pyrimidine nucleobase.

“Primer” means a short double-stranded nucleic acid sequence that has a 3′-OH terminus at which a DNA polymerase can begin synthesis of a nucleic acid chain.

“Incorporation” and “incorporation event”, when used herein to refer to interaction between an immobilized complex and an NTP, mean that the phosphate from the NTP is cleaved, leaving a nucleobase, which becomes incorporated into the primer nucleic acid of the immobilized complex.

To “sequence” means to determine the type and order of successive nucleobases along a strand of a nucleic acid. To “genotype” means to determine that some of the sequence of a part of a nucleic acid is essentially the same as that of a known gene.

“Entrapped” means unable to be carried away by diffusion or solution flow.

The methods of the present invention may be accomplished using rapid fluidic switching in a flow cell. Preferably the flow cell is fabricated with micron-sized features. The flow cell includes three or more, columns that intersect at a junction. In some embodiments, the flow cell can include four or more columns. The use of four or more columns intersecting at a junction in electrophoretic separation of solution components is disclosed, e.g., in U.S. Pat. Nos. 6,033,546; 6,010,608; 6,010,607; 6,001,229; and 5,858,195, the disclosures of which are hereby incorporated herein by reference in their entirety.

The chemical reaction of a single labeled molecule with a surface-bound reagent is detected by (a) binding the reagent molecule to a surface that is within a fluidic flow cell (or that is later introduced into a fluidic flow cell); (b) introducing at least two flowing solutions into the flow cell, wherein at least one solution comprises labeled molecules that can react with the reagent, and at least one solution comprises buffer with no detectable label, and wherein the two solutions exist at different locations within a single channel or flow chamber at any time; (c) switching the location of the flowing solutions with respect to the surface-bound reagent, or the location of the surface-bound reagent with respect to the flowing solutions, so as to immerse the surface-bound reagent molecule in a solution comprising labeled reactive molecules; (d) after a short interval of time that is less than that for which the label is bound or entrapped by the surface-bound reagent (Δt), quickly switching the location of the flowing solutions with respect to the surface-bound reagent, or the location of the surface-bound reagent with respect to the flowing solutions, so as to immerse the surface-bound reagent molecule in a solution comprising buffer with no labeled molecules, wherein the time taken to switch is less than Δt; (e) simultaneous to the flow or location switching in step (d), switching on the illumination light source, or switching on the process of collecting light, or switching on the process of detecting light, or switching the trajectory of any label that is or was bound or entrapped by the surface-bound reagent so that it now passes within the zone of illumination and detection; (f) collecting and detecting or imaging for a time greater than Δt the characteristic light emitted by a label.

A single chemical reaction between a labeled molecule and a surface-bound reagent can be detected by detecting the characteristic light such as fluorescence from the label in step (f). In order to detect a series of chemical reactions with a surface bound reagent, the process of steps (c) through (f) would be repeated, provided that the time Δt is not infinitely long, or provided that other steps are enacted to first effectively remove the label from the surface-bound reagent. The illumination and detection zone may encompass the surface-bound reagent, or it may be at a location within the buffer solution downstream from the surface-bound reagent, so as to avoid irradiation of the surface-bound reagent.

The methods of the present disclosure involve detecting individual labeled molecules in solution. Many variations in the methods are possible. Therefore, the preferred embodiments disclosed herein are intended as illustrative, and not to limit the scope of the invention. In particular, to one skilled in the art of optical instrumentation, it would be straightforward to extend the disclosed method to an embodiment in which an array of surface-bound reagents and an imaging camera are used to resolve and simultaneously detect the chemical reactions at a multiplicity of surface-bound reagents. Also, some preferred embodiments include the application of the disclosed method to the detection of a series of chemical reactions in which the nucleobases of labeled NTP molecules become incorporated into the DNA of a surface-bound enzyme-DNA complex with release of the labels, as would occur in a scheme for DNA sequencing by synthesis. The examples consider the case in which the label is covalently bound to the gamma phosphate of the NTP and in which the label becomes entrapped by the enzyme-DNA complex for a time Δt of about 1.5 milliseconds, before the enzyme conformation changes and the label and pyrophosphate moiety are released into solution. In some embodiments, the time Δt may differ from 1.5 milliseconds. For example, Δt may be enlarged by carrying out the reaction at a reduced temperature or modifying the enzyme used in an enzymatic reaction.

The present invention also provides a system for optically detecting the chemical reaction of a labeled molecule with a surface-bound reagent, wherein the chemical reaction involves at least one step in which the label of the labeled molecule is bound or stereo-chemically entrapped by the reagent, at least for a short interval of time Δt exceeding about 100 microseconds, comprising

a. a surface to which is bound a single reagent molecule, or an array of single reagent molecules at resolvable locations, said surface being contained within a flow cell;

b. an illumination zone;

c. a light source for illuminating said illumination zone;

d. an optical detector for detecting light emitted by a label on said labeled molecule;

e. at least two flowing solutions, wherein at least one solution comprises labeled molecules that can react with said surface-bound reagent, and at least one solution comprises buffer with no detectable label, and wherein the two solutions exist at distinct locations within said flow cell;

f. a means for switching at least one of the flow of said solutions, the physical location of the surface of said surface-bound reagent, to immerse the surface-bound reagent molecule in only one solution at a time; a means for switching on and off at least one of the light source, the detection of light, or the relative location of the zone of illumination-and-detection and the surface of the surface-bound reagent, so that labels that pass nearby to the surface either pass through said zone or do not pass through said zone; and

g. a circuit for effectively synchronizing said switching from part (c) with said switching from part (d) and for appropriately setting the times between said switchings.

The means for switching the flow of solutions can be, for example, an electronic switch that switches the voltages applied at the reservoirs of the solution of buffer and the solution of labeled reactant molecules. The means for switching the location of the supported reagent can be a position-adjustable laser trap or an adjustable mechanical micromanipulator such as a piezo-electrically controlled fiber tip.

The means for switching the light source can be an optical shutter. The means for switching the detection of light can be an electronic switch to alter the bias voltage of an optical detector. The means for altering the relative location of the zone of illumination-and-detection and the surface of the surface-bound reagent can be the same as the means for switching the flow, or the same as the means for switching the location of the supported reagent.

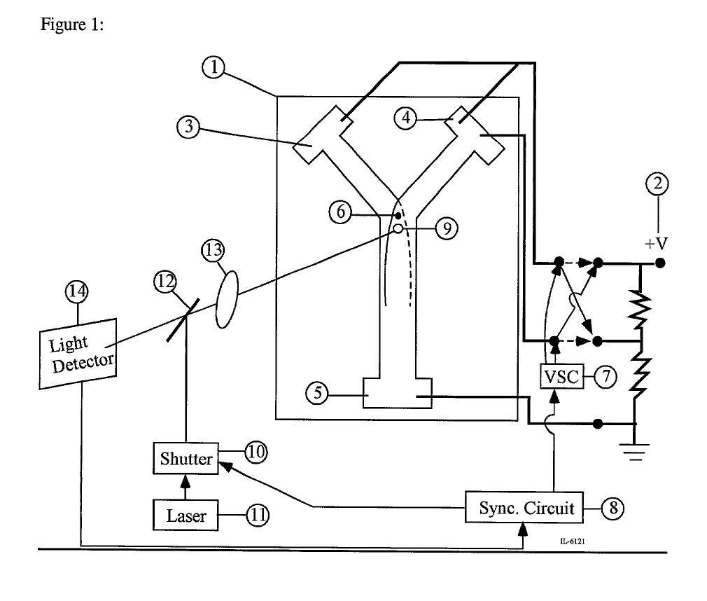

A preferred embodiment is illustrated in schematic form in FIG. 1. A micron-sized flow cell (1) with at least three fluid reservoirs and channels intersecting at a junction is connected to a voltage source (2) for producing electro-osmotically driven flows. The voltage drives a buffer solution from a buffer fluid reservoir (3) and a solution of labeled NTP molecules from a labeled-NTP reservoir (4) towards a waste reservoir (5). An affixed enzyme-DNA complex (6) is located near the junction immediately downstream from the intersection of the channels and in the center of the channel that is connected to the waste reservoir. An electronic voltage switching circuit (7) is used to switch the driving voltages that are applied to the reservoirs between two states, illustrated by solid and dashed lines. In the first state (shown by solid lines), a higher voltage is applied to the labeled-NTP reservoir, thereby causing a larger volumetric flow from this reservoir, which in turn causes the affixed enzyme-DNA complex to be immersed in a solution of labeled-NTP molecules. In the second state (shown by dashed lines), a higher voltage is applied to the buffer reservoir, thereby causing the affixed enzyme-DNA complex to be immersed in a solution of buffer, with no labels present (unless a single label had been retained at the affixed complex).

The solution of labeled-NTP molecules contains about 10⁵ M of each of the four types of NTPs, and each type of NTP in the solution is labeled by a fluorescent moiety of different color. Because the concentration of each type of NTP is high, incorporation of a complementary nucleobase onto the primer of the enzyme-DNA complex is likely to initiate almost immediately. A timing and synchronization circuit (8) controls the voltage switching circuit so that the voltage levels and flow remain in the first state for a period of time that is approximately 0.5 milliseconds or shorter. If incorporation of a nucleobase is initiated, then the enzyme-DNA complex will entrap the label of the NTP that is being incorporated for at least 1.5 milliseconds. The voltage levels and flow are switched from the first into the second state within a period of time that is 0.5 milliseconds or shorter, and ideally within a time as short as possible. The affixed enzyme-DNA complex is now immersed in buffer solution, with no labels present except for the single label that would be present if incorporation had become initiated. An illumination and single labeled-molecule detection zone (9) is located immediately downstream from the affixed complex. A laser shutter (10), also controlled by the timing and synchronization circuit (8), is opened within a time shorter than 0.5 milliseconds following the immersion of the complex in buffer solution. The opening of the shutter allows the beam from a laser (11) to impinge upon the illumination zone. In FIG. 1, an epi-illumination system is depicted. The laser beam enters at a dichroic beam splitter (12) and through a microscope objective lens (13), which is also used to collect light from the illumination zone. However, optical configurations other than epi-illumination may also be used. The light collected from the illumination zone enters a light detection system (14), which may contain spatial and spectral filters and a sensitive photon detector or camera for discerning the characteristic signal from a single label passing through the illumination zone. The shutter (10) may be positioned within the light detection system, and the illumination zone may be continually irradiated, but the light detection system (14) is irradiated with collected light only when the shutter is open. Alternatively, the operation of the detection system can be shuttered, for example, by modulating the electronics or voltage supply to the light detector, i.e., an effective shutter would be contained within the electronics of the light detection system.

The period of time that the shutter remains opens must be long enough to allow the label to be released from the affixed complex. In the present example, the minimum time that the enzyme entraps the label is 1.5 milliseconds, and hence the shutter should remain open for a time longer than this, unless a released label is detected earlier. In this preferred embodiment, the period of time that the shutter remains open is set by the timing and synchronization circuit (8) to be 15 milliseconds, but it is terminated sooner if a label is detected and a signal is sent from the light detection system to the timing and synchronization circuit. If a label is detected, the light detection system also ascertains the identity of the label that is detected, according to the color (or lifetime, or other characteristic) of the detected photons, and hence the identity of the nucleobase that had just become incorporated is known.

As soon as the shutter is closed, the timing and synchronization circuit (8) directs the voltage switching circuit (7) to switch back into the first state (affixed complex immersed in labeled-NTP solution) for another period of time that is approximately 0.5 milliseconds or shorter, and the process is cycled.

Molecular diffusion in solution can determine the flow conditions that are required in order to cleanly switch from a first state in which the affixed complex is immersed in a solution of labeled NTPs of concentration about 10⁻⁵ M to a second state in which the immobilized complex is immersed in a solution of buffer with no free labeled molecules. The diffusional mixing of the labeled-NTP solution and the buffer solution initiates at the apex of the “Y” junction of the micro-capillaries and becomes more complete as the solutions flow towards the waste reservoir. Therefore, preferably the complex is affixed in close proximity to the apex of the junction. Also, preferably the solution flow should be electro-osmotically driven to provide minimal diffusional mixing, and the velocity of the flowing solutions should be as fast as possible, while still remaining slow enough to provide adequate signal integration time for detecting a label in the illumination zone. Also, the solutions may contain a viscous component such as glycerol to slow down diffusional mixing. Also, the buffer and labeled-NTP solutions may be chosen to be immiscible and the labeled-NTPs may be insoluble in the buffer solution.

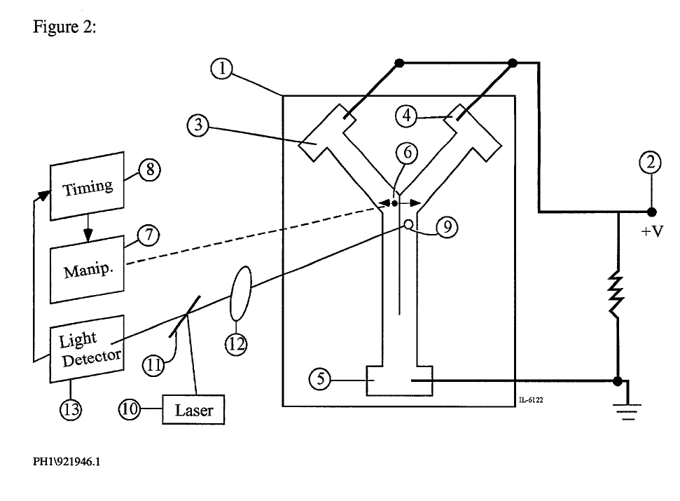

Another preferred embodiment is illustrated in schematic form in FIG. 2. In this embodiment, the synchronization of the switching of the concentration of solution in which the complex is immersed, with the switching of the location that is downstream from the complex, occurs automatically. In practice, it is not necessary to switch the laser illumination intensity. However, the set up still involves synchronization because the level of irradiation at the location surrounding or downstream from the surface-bound complex is effectively switched, as the location of the complex is switched.

In the embodiment illustrated in FIG. 2, a flow cell (1) with at least three fluid reservoirs and channels intersecting at a junction is connected to a voltage source (2) for producing electro-osmotically driven flows. The voltage drives a solution of labeled-NTP molecules from a labeled-NTP reservoir (3) and a buffer solution from a buffer fluid reservoir (4) at steady and equal volumetric flows towards a waste reservoir (5). An enzyme-DNA complex (6) is bound to a surface, such as the surface of a microbead or fiber, which is introduced into the flow cell and held with laser tweezers or a piezoelectric micromanipulator (7) within the channel that is connected to the waste reservoir and immediately downstream from the apex of the junction of the other two channels. The complex within the flow cell can be quickly moved between two locations by controlling the focal position of the laser tweezers or the extension of the micromanipulator. In the first location (which is that depicted in FIG. 2) the surface-bound enzyme-DNA complex is immersed in a solution of labeled-NTP molecules. In the second location, the enzyme-DNA complex is shifted within the flow cell so that it is immersed in a solution of buffer, with no labels present.

The solution of labeled-NTP molecules contains about 10⁵M of each of the four types of NTPs, and each type of NTP in the solution is labeled by a fluorescent moiety of different color. Because the concentration of each type of NTP is high, incorporation of a complementary nucleobase onto the primer of the enzyme-DNA complex is likely to initiate almost immediately. A timing circuit (8) controls the laser tweezers or micromanipulator so that the location of the surface and complex remains in the first state for a period of time that is approximately 0.5 milliseconds or shorter. If incorporation of a nucleobase is initiated, then the enzyme will entrap the label of the NTP that is being incorporated for at least 1.5 milliseconds. The timing circuit signals the laser tweezers or micromanipulator to switch the location of the surface and complex from the first state into the second state within a period of time that is 0.5 milliseconds or shorter, and ideally as quickly as possible. The surface-bound enzyme-DNA complex is now immersed in buffer solution, with no labels present except for the single label that would be present if incorporation had become initiated. An illumination and single labeled-molecule detection zone (9) is located within the buffer solution immediately downstream from the second-state location of the complex. In FIG. 2, an epi-illumination system for single-molecule detection is depicted. The beam from a laser (10) enters at a dichroic beam splitter (11) and through a microscope objective lens (12), which is also used to collect light from the illumination zone onto the light detection system (13), which may contain spatial and spectral filters and a sensitive photon detector or camera for discerning the characteristic signal from a single label passing through the illumination zone. However, optical configurations other than epi-illumination may also be used.

The surface of the surface-bound complex remains at the location of the second state for a time sufficient to allow the label to be released from the immobilized complex. In the present example, the minimum time that the enzyme entraps the label is 1.5 milliseconds, so the surface should remain at the second location for a time longer than this, unless a released label is detected earlier. In this preferred embodiment, the period of time that the surface remains at the second location is set by the timing circuit (8) to be 15 milliseconds, but it is terminated sooner if a label is detected and a signal is sent from the light detection system to the timing and synchronization circuit. If a label is detected, the light detection system also ascertains the identity of the label that is detected, according to the color (or lifetime, or other characteristic) of the detected photons, and hence the identity of the nucleobase that had just become incorporated becomes known. The timing circuit (8) then directs the laser tweezers or micromanipulator (7) to switch the surface back into the first location (with the surface-bound complex immersed in labeled-NTP solution) for another period of time that is approximately 0.5 milliseconds or shorter, and the process can be repeated one or more times.

The methods disclosed herein are useful for optically detecting single events in which the nucleobase from a fluorescently labeled NTP becomes incorporated onto a strand of DNA by the action of a polymerase enzyme, and so could be used in a method for sequencing DNA by synthesis. According to the present invention, with regard to DNA sequencing by synthesis, the chemical reaction of the incorporation of a nucleobase into a surface-bound enzyme-DNA complex can be detected either with or without the use of quenching moieties on the NTPs because fluorescently labeled NTPs that have not become incorporated do not pass through the illumination zone at the time of detection.

It will be recognized by those skilled in the art that the methods and devices disclosed herein can utilize labels that are excited by a variety of wavelengths of light. Other embodiments will be apparent to one skilled in the art.

What is claimed is:

1. A method for optically detecting labeled molecules that have participated in a chemical reaction with a reagent supported on a surface, comprising:
a) providing a flow cell:
b) providing within said flow cell a solid support having a surface;
c) supporting at least one reagent molecule to said surface;
d) introducing at least two flowing solutions into said flow cell, wherein at least one solution comprises a labeled molecule that can contact said reagent molecule, and at least one solution comprises buffer with no detectable labels, and wherein the at least two solutions are at different locations within the flow cell at any time;
e) immersing the supported reagent in a solution comprising labeled molecules;
f) switching or directing the flowing solutions with respect to the supported reagent, or switching the location of the supported reagent with respect to the flowing solutions, to immerse the supported reagent in the solution comprising buffer with no labeled molecules;
g) providing a light source for illuminating an illumination zone within the buffer solution;
h) providing a detector for detecting light emitted from the illumination zone;
i) substantially simultaneously to said switching or directing the flowing solutions with respect to the supported reagent, or switching the location of the supported reagent with respect to the flowing solutions, switching at least one of said light source, detector, or location of said supported reagent to cause the label of a labeled molecule which has reacted with said supported reagent to pass through said illumination zone; and
j) detecting light emitted at said illumination zone to detect the presence of one or more labeled molecules.

2. A method according to claim 1 wherein a single labeled molecule is detected by said step of detecting light emitted at said illumination zone.

3. A method according to claim 1 wherein a chemical reaction is detected by detecting the presence of labeled molecules that have participated in said chemical reaction.

4. A method according to claim 1 wherein a single chemical reaction is detected by detecting the presence of a single labeled molecule that has participated in said chemical reaction by said step of detecting light emitted at said illumination zone.

5. A method according to claim 4 wherein the concentration of said labeled molecules is above 10⁻⁸ M.

6. A method according to claim 4 wherein the concentration said labeled molecules is above 10⁻⁷ M.

7. A method according to claim 4 wherein the concentration of said labeled molecules is above 10⁻⁶ M.

8. A method according to claim 4 wherein the concentration of reactant labeled molecules is above 10⁻⁵ M.

9. A method according to claim 1 wherein the label is a fluorescent label.

10. A method according to claim 5 wherein the supported reagent is a complex comprising a surface-bound polymerase enzyme and a nucleic acid or a supported nucleic acid and a polymerase, and the solution of labeled reactive molecules comprises at least one type of fluorescently labeled NTP molecule with no quenching moiety.

11. A method according to claim 10 wherein said fluorescent labels are attached to the beta or gamma phosphate of the NTP.

12. A method according to claim 1 wherein two or more distinguishable types of labels are used to label two or more different types of reactive molecules.

13. A method of claim 1 wherein optical detection of the labels includes identifying the labels by a property selected from the group consisting of color of excitation light or emission light, fluorescence lifetime, fluorescence brightness, electrophoretic mobility, location of detection, or time of detection.

14. A method according to claim 3 wherein an array of supported reagents is used and wherein optical detection of the reactions are separately accomplished for each reagent of the array.

15. A method of claim 3 wherein a series of chemical reactions is detected by repeating said steps of introducing said at least two flowing solutions into said flow cell, immersing said supported reagent in a solution comprising labeled molecules, switching or directing the flowing solutions with respect to the supported reagent, or switching the location of the supported reagent with respect to the flowing solutions, switching at least one of said light source, detector, or location of said supported reagent to cause the label of a labeled molecule which has reacted with said supported reagent to pass through said illumination zone; and detecting light emitted at said illumination zone to detect the presence of one or more labeled molecules.

16. A method of claim 15 wherein the time interval between successive chemical reactions is controlled by controlling the time between successive repetitions.

17. A system for optically detecting the chemical reaction of a labeled molecule with a surface-bound reagent, wherein the chemical reaction involves at least one step in which the label of the labeled molecule is bound or stereo-chemically entrapped by the reagent, at least for a short interval of time Δt exceeding about 100 microseconds, comprising:
a. a surface to which is bound a single reagent molecule, or an array of single reagent molecules at resolvable locations, said surface being contained within a flow cell;
b. an illumination zone;
c. a light source for illuminating said illumination zone;
d. an optical detector for detecting light emitted by a label on said labeled molecule.;
e. at least two flowing solutions, wherein at least one solution comprises labeled molecules that can react with said surface-bound reagent, and at least one solution comprises buffer with no detectable label, and wherein the two solutions exist at distinct locations within said flow cell;
f. a means for switching at least one of the flow of said solutions, the physical location of the surface of said surface-bound reagent, to immerse the surface-bound reagent molecule in only one solution at a time;
g. a means for switching on and off at least one of the light source, the detection of light, or the relative location of the zone of illumination-and-detection and the surface of the surface-bound reagent, so that labels that pass nearby to the surface either pass through said zone or do not pass through said zone; and
h. a circuit for effectively synchronizing said illuminating said illumination zone with said switching at least one of the flow of said solutions or the physical location of the surface of said surface-bound reagent, to immerse the surface-bound reagent molecule in only one solution at a time.
